(19) United States
(12) Patent Application Publication
(10) Pub. No.: US 2025/0172533 A1
(43) Pub. Date: May 29, 2025

(54) RAPID DETECTION AND PREDICTION OF FUNCTIONAL CHARACTERISTICS OF DAIRY POWDERS USING NEAR-INFRARED SPECTROSCOPY

(71) Applicant: Regents of the University of Minnesota, Minneapolis, MN (US)

(72) Inventors: Kumar Mallikarjunan, Minneapolis, MN (US); Muath Alessa, Minneapolis, MN (US); Sonali Raghunath, Minneapolis, MN (US); Priyanshi Chaturvedi, Minneapolis, MN (US)

(21) Appl. No.: 18/950,842

(22) Filed: Nov. 18, 2024

Related U.S. Application Data
(60) Provisional application No. 63/602,867, filed on Nov. 27, 2023.

(51) Int. Cl. G01N 33/04; G01N 21/359

(52) U.S. Cl. CPC G01N 33/04; G01N 21/359

(57) ABSTRACT

A method of predicting a value for a functional property of a dairy powder includes placing a sample of the dairy powder in a near-infrared spectrophotometer and using the near-infrared spectrophotometer to determine intensities of different wavelengths of light reflected from the sample within a near-infrared band. The intensities are then used to determine a predicted value for the functional property.

### CROSS-REFERENCE TO RELATED APPLICATION

The present application is based on and claims the benefit of U.S. provisional patent application Ser. No. 63/602,867, filed Nov. 27, 2023, the content of which is hereby incorporated by reference in its entirety.

### BACKGROUND

The functional properties of dairy powders are variable and challenging to control, and this can lead to undesirable functional properties after subjected to storage or transportation conditions due to various internal and external environmental factors. The methods available to determine functional properties are time-consuming, destructive, and there is a need for the development of rapid non-destructive analytical techniques.

The discussion above is merely provided for general background information and is not intended to be used as an aid in determining the scope of the claimed subject matter. The claimed subject matter is not limited to implementations that solve any or all disadvantages noted in the background.

### SUMMARY

A method of predicting a value for a functional property of a dairy powder includes placing a sample of the dairy powder in a near-infrared Spectrophotometer and using the near-infrared Spectrophotometer to determine intensities of different wavelengths of light reflected from the sample within a near-infrared band. These intensities are then used to determine a predicted value for the functional property.

In accordance with a further embodiment, a dairy powder functional prediction system includes a near-infrared light source providing wavelengths of light across a near-infrared spectrum and a sample holder for holding a sample of a dairy powder. A light sensing assembly is positioned to measure intensities of different wavelengths of light reflected by the dairy powder in the sample holder and a processor, receives the measured intensities and uses the measured intensities to predict a value for a functional property of the dairy powder.

In accordance with a still further embodiment, a method includes producing a plurality of different types of dairy powders and dividing each dairy powder type into a plurality of testing samples. Each testing sample is exposed to a respective temperature and/or humidity for a period of days and near-infrared light reflectance intensities of the exposed testing samples are measured. Respective values for a functional property of each exposed testing sample are determined and the measured reflectance intensities and the determined values for the functional properties are used to construct a model that predicts values for the functional property from measured near-infrared light reflectance.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 provides a tree showing types of dry dairy powders and their ingredients.

FIG. 2 provides a simple block diagram of NIR Flex Buchi.

FIG. 3A provides graphs of spectra for multiple samples without any pretreatment.

FIG. 3B provides the graphs of spectra for the multiple samples of FIG. 3A with standard normal variate pretreatment.

FIG. 4A provides a graph of Q values as a function of number of principal components in the model for foaming capacity.

FIG. 4B provides a graph of SEC/SEP % as a function of number of principal components in the model for foaming capacity.

FIG. 5A provides a graph of the Validation set Predicted residual error sum square of the foaming capacity as a function of the number of principal components in the model.

FIG. 5B provides a graph of the Calibration set Predicted residual error sum square of the foaming capacity as a function of the number of principal components in the model.

FIG. 5C provides a graph of the Validation set standard error of estimation of the foaming capacity as a function of the number of principal components in the model.

FIG. 5D provides a graph of the Calibration set standard error of estimation of the foaming capacity as a function of the number of principal components in the model.

FIG. 6A provides a graph of the Calibration set regression coefficient of foaming capacity as a function of the number of principal components in the model

FIG. 6B provides a graph of the Validation set regression coefficient of foaming capacity as a function of the number of principal components in the model.

FIG. 7 provides a graph of the Validation set BIAS of foaming capacity as a function of the number of principal components in the model.

FIG. 8 provides a graph of predicted foaming capacity values from the model as a function of the actual foaming capacity values of the samples.

FIG. 9A provides graphs of spectra for multiple samples without any pretreatment.

FIG. 9B provides the graphs of spectra for the multiple samples of FIG. 9A with db1 and ncl.

FIG. 10A provides a graph of Q values as a function of number of principal components in the model for foaming stability.

FIG. 10B provides a graph of SEC/SEP % as a function of number of principal components in the model for foaming stability.

FIG. 11A provides a graph of the Validation set Predicted residual error sum square of the foaming stability as a function of the number of principal components in the model.

FIG. 11B provides a graph of the Calibration set Predicted residual error sum square of the foaming stability as a function of the number of principal components in the model.

FIG. 11C provides a graph of the Validation set standard error of estimation of the foaming stability as a function of the number of principal components in the model.

FIG. 11D provides a graph of the Calibration set standard error of estimation of the foaming stability as a function of the number of principal components in the model.

FIG. 12A provides a graph of the Calibration set regression coefficient of foaming stability as a function of the number of principal components in the model.

FIG. 12B provides a graph of the Validation set regression coefficient of foaming stability as a function of the number of principal components in the model (right graph).

FIG. 13 provides graph of the Validation set BIAS of foaming stability as a function of the number of principal components in the model.

FIG. 14 provides a graph of predicted foaming stability values from the model as a function of the actual foaming stability values of the samples.

FIG. 15A provides graphs of spectra for multiple samples without any pretreatment.

FIG. 15B provides the graphs of spectra for the multiple samples of FIG. 9(a) with standard normal variant.

FIG. 16A provides a graph of Q values as a function of number of principal components in the model for solubility.

FIG. 16B provides a graph of SEC/SEP % as a function of number of principal components in the model for solubility.

FIG. 17A provides a graph of the Validation set Predicted residual error sum square of the solubility as a function of the number of principal components in the model.

FIG. 17B provides a graph of the Calibration set Predicted residual error sum square of the solubility as a function of the number of principal components in the model.

FIG. 17C provides a graph of the Validation set standard error of estimation of the solubility as a function of the number of principal components in the model.

FIG. 17D provides a graph of the Calibration set standard error of estimation of the solubility as a function of the number of principal components in the model.

FIG. 18A provides a graph of the Calibration set regression coefficient of solubility as a function of the number of principal components in the model.

FIG. 18B provides a graph of the Validation set regression coefficient of solubility as a function of the number of principal components in the model.

FIG. 19 provides graph of the Validation set BIAS of solubility as a function of the number of principal components in the model.

FIG. 20 provides a graph of predicted solubility values from the model as a function of the actual solubility values of the samples.

FIG. 21 provides a block diagram of spectrophotometer system in accordance with one embodiment.

FIG. 22 provides a flow diagram of a method constructing a predictive model in accordance with one embodiment.

FIG. 23 provides a flow diagram of a method of predicting a functional property of a dairy powder from near infrared spectra reflected by the powder.

### DETAILED DESCRIPTION

In the present description, several abbreviations are used. The abbreviations and the terms they represent are as follows:

SEC: Standard Error of Calibration;

SEP: Standard Error of Prediction;

Q Value: Proprietary Quality factor;

C-SET PRESS: Calibration Set sum of the Predicted Residual Error Square;

V-SET PRESS: Validation Set sum of the Predicted Residual Error Square;

SNV: Standard Normal Variate;

BCAP (db1): 1st Derivative analysis of principle components;

ncl: normalized by closure;

PC: Principal Components;

Bias: Deviation between the values of predicted and actual.

In recent years, the usage of dairy powders as ingredients in formulations has increased due to consumers' demand for highly nutritious products. There are various types of dairy powders available in the market, which are classified based on separation or the concentration process as: whey protein concentrates, whey protein isolates, milk protein concentrates, milk protein isolates, and nonfat dry milk.

In accordance with one embodiment, FT-NIR (Fourier Transform near Infrared Spectroscopy) technology is used as a method for rapid prediction of functional properties of a dairy powder.

In accordance with one embodiment, dairy powders of different types are divided into testing samples and each sample is stored at different combinations of humidity conditions (32.8%, 66.4%, 100%), temperature conditions (4° C., 22°° C., 40° C.) and durations (3 and 10 days). After storing the samples, values for the functional properties: solubility, foaming capacity, and foaming stability are determined for each testing sample using traditional time-consuming analysis methods. Using FT-NIR, spectral data is collected for each testing sample after storage. In accordance with one embodiment, the spectral data of these samples are collected from near-infrared regions (4000 cm⁻¹ to 1250 cm⁻¹). The values for the functional properties and the spectral data are applied to chemometric analytical algorithms to identify significant spectral bands (principal components) that correlate to the functional properties and the relationships between the intensities of those bands and the values of the functional properties.

In accordance with one embodiment, a separate prediction model is determined for each functional property (solubility and foaming) and each prediction model works with five different types of dairy powders:
Whey protein concentrate
Whey protein isolate.
Milk protein concentrate.
Milk protein isolate
Nonfat dry milk

The embodiments identify which of thousands of spectral bands in the near infrared range are principal components in predicting a dairy powder's solubility and foaming characteristics and determine the relationships between the intensities of the identified spectral bands and the values for the solubility and foaming characteristics. In accordance with one embodiment, the relationships were identified using partial least squares regression, and various pretreatments were employed to eliminate noise. From the analysis of the results, the data sets for solubility and foaming characteristics produced a good prediction model with 11 PC (Principal components) for foaming capacity, 7 PCs for foaming stability, and 15 PCs for solubility. The regression coefficient R² for all the predicted models >0.9 for predicted vs. original property, demonstrating the robustness of the models developed.

### Dairy Powders

Dairy powders are adaptable ingredients in the market. These powders are produced by the dehydration of milk and milk products through spray drying or evaporation, making it easier for transportation and shipping. Dairy powders are used in a wide range of products, including cheese, bakery, confectionery, infant formulas, healthy and nutritious meal replacers, and chocolates.

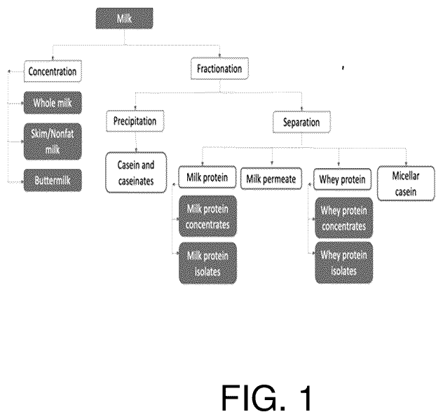

Dairy powders are classified based on composition (protein, fat, lactose, and minerals) and process technology for the elimination of specific components of the serum phase including water, lactose, proteins, and fat. In terms of fat composition, the powders are subcategorized as whole milk powder and skim milk powder, also known as non-fat dry milk powder. Based on protein composition and concentration, they are classified into whey protein concentrates and isolates (WPC & WPI), milk protein concentrates and isolates (MPC & MPI), where isolates consist of >90% protein, while concentrates contain 40-86% protein. For long-term storage, transportation and distribution, milk powders are usually dehydrated, and these dairy powders need to retain their functional properties after transport to be used as an ingredient. FIG. 1 represents a summary of various dairy powders manufactured from milk.

Converting milks into powders can cause a loss of functionality, such as caking, impaired rate of hydration, loss in solubility, and poor reconstitution ability. In addition, changes in storage conditions (temperature and relative humidity), composition, capillary interactions within particles, and migration of cohesive chemical components to the surface of powder particles impact the functionality of dairy powders.

One of the challenges in the dairy industry is the lack of rapid and efficient evaluation of dairy powder functionality before hydration. Due to the conventional methods available, there are always delays in understanding the influence of storage, transportation, and other environmental parameters in dairy powder functionality and where it can be ideally used as an ingredient.

### Predictive Model Construction

In accordance with one embodiment, a separate predictive model is constructed for each functional property of dairy powders to be predicted. Each predictive model is constructed by first exposing samples of the dairy powders to different storage environments and then performing traditional analysis and spectral analysis on the exposed samples. The resulting data is then used to construct the predictive model.

### Sample Exposure

Each model is constructed using samples of five different types of diary powders: Whey Protein Isolate 90 (WPI90), Whey Protein Concentrate 80 (WPC80), Milk Protein Isolate 90 (MPI90), Milk Protein Concentrate 85 (MPC85), and Nonfat Dry Milk (NFDM) that are exposed to different storage environments. Each dairy powder type is divided into a plurality of test samples, with each test sample being exposed to a respective storage environment consisting of a combination of humidity and temperature conditions for selected storage/exposure periods. The different humidity, temperature and exposure periods impact the functional properties of the dairy powders in different ways. As a result, the exposed samples provide a broad range of solubilities and foaming properties. In accordance with one embodiment, the five different sets of commercial dairy powders, are stored at different humidity (32.8%, 66.4%, 100%) and temperatures (4° C., 22° C., 40° C.) for 3 and 10 days. For example, three samples of whey protein concentrate are stored at 32.8% relative humidity and 4° C. for three days while another three samples of whey protein concentrate are stored at 32.8% relative humidity and 4° C. for ten days. Thus, in this embodiment, each dairy powder type is divided into fifty-four testing samples so that three samples of each dairy powder type are exposed to each of the eighteen exposure environments, where each exposure environment consists of a unique combination of relative humidity, temperature and duration (3 relative humidity values*3 temperature values*2 duration values=18 unique exposure environments).

The humidity conditions were maintained at 100% using water, 66.4% using saturated potassium iodide solution, and 32.8% using saturated magnesium chloride solution. The exposure design is presented in Table 1 below with Whey Protein Isolate 90 (WPI90), Whey Protein Concentrate 80 (WPC80), Milk Protein Isolate 90 (MPI90), Milk Protein Concentrate 85 (MPC85), and Nonfat Dry Milk (NFDM). All the samples are prepared in triplicates. These samples were used as a training set for NIR analysis.

TABLE 1

| Temp | RH | WPI 90 | WPC 80 | MPI 90 | MPC 85 | NFDM |
|---|---|---|---|---|---|---|
| 4° C. | 100 | 4WPI100 | 4WPC100 | 4MPI100 | 4MPC100 | 4NFDM100 |
| | 66 | 4WPI66 | 4WPC66 | 4MPI66 | 4MPC66 | 4NFDM66 |
| | 33 | 4WPI33 | 4WPC33 | 4MPI33 | 4MPC33 | 4NFDM33 |
| 22° C. | 100 | 22WPI100 | 22WPC100 | 22MPI100 | 22MPC100 | 22NFDM100 |
| | 66 | 22WPI66 | 22WPC66 | 22MPI66 | 22MPC66 | 22NFDM66 |
| | 33 | 22WPI33 | 22WPC33 | 22MPI33 | 22MPC33 | 22NFDM33 |
| 40° C. | 100 | 40WPI100 | 40WPI100 | 40MPI100 | 40MPC100 | 40NFDM100 |
| | 66 | 40WPI66 | 40WPI66 | 40MPI66 | 40MPI66 | 40NFDM66 |
| | 33 | 40WPI33 | 40WPI33 | 40MPI33 | 40MPI33 | 40NFDM33 |

(RH = Relative humidity (%))

### Conventional Methods of Analysis

The exposed samples are tested based on conventional methods to determine values for functional properties of the exposed samples such as: solubility, foaming stability, and foaming capacity.

### Solubility of Dairy Powders

Solubility is a critical functional characteristic since many properties depend on how well the samples are solubilized in water. During storage, increased protein-protein hydrophobic interactions lead to the formation of insoluble casein aggregates with time and temperature.

In accordance with one embodiment, the solubility of the exposed testing samples is determined using the following method. First, 6% protein solutions are mixed at 22° C. for 1 hour. The solution is transferred to 50 ml centrifuge tubes and centrifuged at 700 g for 10 minutes. After centrifugation, the supernatant is transferred to another tube to determine its weight. The total solids of the supernatant are determined using a CEM Smart Turbo Moisture (CEM Corporation, Charlotte, NC) analyzer, which operates based on microwave technology. The following equation is used to calculate the sample's solubility.

Weight of the supernatant=‘A’ grams
Percentage solids of the supernatant=‘B’ %
Total soluble solids in the supernatant=(A*B)/100=C
% solubility=(Total soluble solids in the supernatant (C)/Grams of initial powder)*100

For solubility, from the data, it could be observed that NFDM stored at 66%/22° C./3 days and 33%/4° C./10 days had the highest solubility while MPC85 (33%/40° C./10 days) and MPC (100%/4° C./3 days) had the lowest solubility. Ranking the solubility of powders from highest to lowest, NFDM is highly soluble and is followed by WPC˜WPI>MPI˜MPC.

### Foaming Capacity and Stability of Dairy Powders

The formation of foams occurs due to the unfolding of the hydrophobic regions of proteins and air interactions. The whipping process for the foam formation triggers the protein molecules to unfold and decrease the surface tension between the air and water interface. The presence of non-micellar casein can lead to increased absorption and higher foams in high-protein powders. The strength of the interfacial layer is determined by understanding the foaming stability of the powders.

The foaming characteristics of proteins depend on several parameters, such as type of protein concentration, pH, and whipping conditions such as time, volume, bowl, and speed. The processing conditions include composition, heat, and pH during the manufacturing of the protein solution. Additionally, it is important to note that the methodology used to evaluate foaming characteristics may differ based on the instrument used to whip the foam. Hence, results may vary from one experiment to another.

In accordance with one embodiment, the foaming capacity and foaming stability of the exposed testing samples are determined using the following method. First, 12% sample solutions are prepared. To create the foam, the solution is whipped for two minutes and the whipped solution is poured into a 250 ml measuring cylinder. The initial liquid and foam levels are recorded. After 30 minutes, the final liquid and foam levels are recorded to calculate foaming capacity and stability, as shown below.

The volume of foam (ml)=Total volume of the solution with foam−Total liquid volume
Foaming capacity=Initial foam volume (ml)/g of protein in the solution
Foaming stability (%)=(Final foam volume/Initial foam volume)×100

From the analysis, the highest foaming capacity to the lowest was achieved for NFDM and WPC80. According to our analysis, NFDM>MPC˜MPI>WPI>WPC from the highest to the lowest foaming capacity and foaming stability. The difference in results could be potentially due to the difference in the measurement method and the effect of storage treatments on the sample for analysis.

### Spectral Data Collection

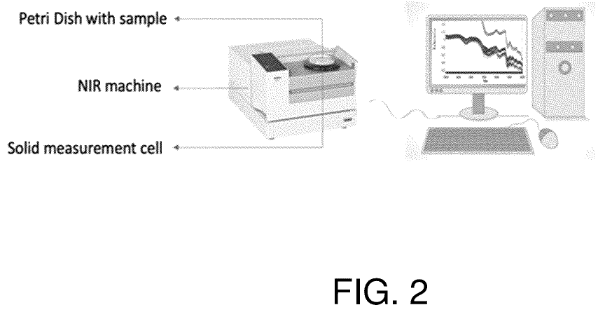

Near IR spectral data is collected from each of the exposed testing samples. In accordance with one embodiment, the spectral data is collected using a NIR-Flex (Buchi Labortechnik, Flawil, Switzerland) with a solid's measurement cell, as shown in FIG. 2.

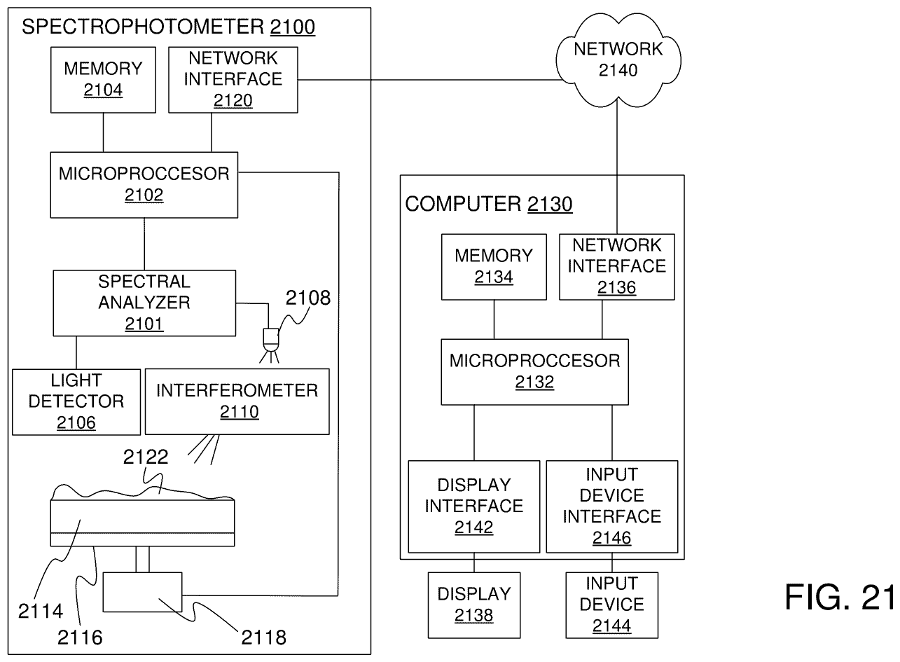

Petri dishes with samples are placed on the solid measurement cell of the NIR machine to collect spectral data. Details of the data collection are discussed below with reference to FIG. 21.

### Data Correlation

The values for the functional properties obtained through the conventional methods and the spectral data are correlated to produce the predictive models for each functional property.

The values for the functional properties and the spectral data are used to construct predictive models, one for each functional property, that are able to predict a value for a functional property based on spectral data. This involves identifying which spectral bands are significant to predicting the functional properties and how the intensities of those significant bands relate to the values of the functional properties. In accordance with one embodiment, the identification of the significant bands and the relationships between the intensities and the values of the functional properties are determined using Chemometric Software such as NIRCal 5.2 Chemometric Software (BUCHI Labortechnik) and statistical methods like Partial Least Squares (PLS) and Principal Component Analysis (PCA). The predictive models are optimized to achieve the highest correlation coefficient and minimize the standard error by adjusting pretreatments, spectral regions, and the number of principal components (PC). Pretreatments such as normalization, derivatives, and standard normal variate are applied to reduce unwanted variations.

Chemometrics is mainly used to reduce data complexity and enhance the models' accuracy and predictability based on spectral datasets. The steps of chemometric spectral analysis include 1. Preprocessing the spectral data for reducing interferences: scattering, baseline drift, path length variation, overlapped bands 2. Selecting optimal variables from a huge spectral data set to develop a reliable model 3. Validation and interpretation of the results.

### Preprocessing or Pretreatment of NIR Spectral Data

Generally, food samples like dairy powders are affected by various physical interventions or instrumental interference by scattering. The NIR spectral data shows the differences associated with searched differences; however, there are high chances of getting false classifications in the original spectra. At first, the original spectra undergo pretreatment to eliminate unwanted effects. To eliminate these unwanted variations, usage of appropriate data pretreatment methods are applied to reduce the noise and to minimize the influence of these on the prediction model to have robust and reliable data. The most commonly used pretreatment methods include scattering-related methods like standard normal variate and normalization, which are generally used to minimize any effects related to scattering.

The spectra for solids are typically known to have some scattering due to differences in particle size or differences due to the thickness of the glass container used for analysis or differences due to how densely the powders are packed. To minimize any baseline shifts or drifts in the dataset or improve the resolutions of the spectral data derivatives like, Savitzky models were added to the spectral data analysis. Derivatives are used when chemically different substances are studied since it will separate the substances. The absorption peaks are more significant in the first derivative, or any overlapping peaks were better recognized. In the present embodiments, the pretreatments such as normalization, derivatives, and standard normal variate are applied to reduce this scattering effect.

### NIR Calibration for Foaming Capacity

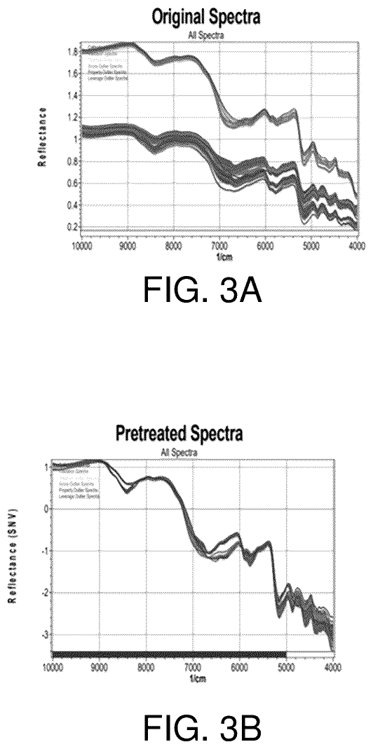

The dairy powder samples tend to have particle size variances due to their nature of processing and spray drying conditions. In solids like powder, scattering is caused due to particle size differences, differences in the thickness of the petri dish, or differences in packing the powder. These differences can potentially create inconsistencies with calibration spectra and result in less model prediction accuracy for dairy powders. Hence, Standard Normal Variant (SNV) wavelength-related pretreatment is applied to reduce the scattering in this dataset. SNV centered each of the original spectra, then scaled to its standard deviation, and only used data from that spectrum. The SNV corrected the y-axis and centered the spectra, minimizing any interferences, as shown in FIGS. 3A and 3B where FIG. 3A show the spectra without and FIG. 3B with pretreatment with the standard normal variant.

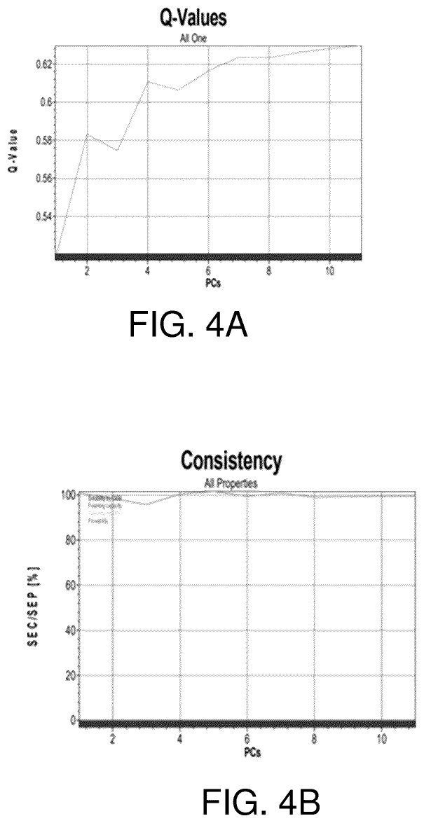

The Principal Components are selected to have a direct influence on the calibration model. From the analysis, 11 PCs were chosen as optimal for calibrating the foaming capacity of dairy powders under study. Furthermore, the Q value, a Proprietary quality factor from Buchi, provided a value of 0.62 for 11 PCs with SNV as pretreatment (FIG. 4A), which served as a quality marker to determine the optimal number of PCs for the dataset. Q value of 0.6 and above is considered acceptable and preferably over 0.8. Also, consistency graphs (FIG. 4B) supported understanding the relationship between SEC and SEP with 11 PCs with a value of 99%, proving the robustness of the models and optimum PCs.

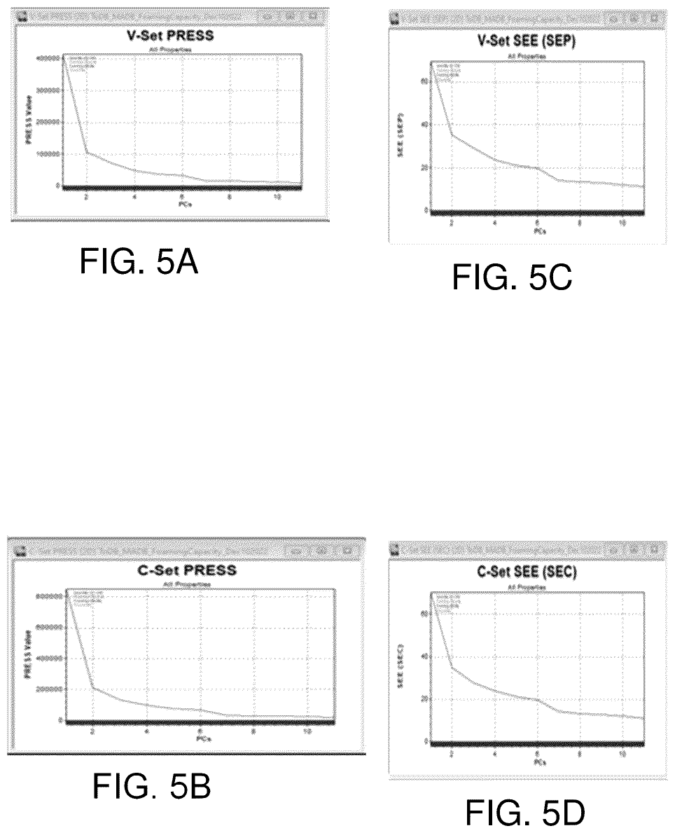

The V-SET and C-SET graphs from FIGS. 5A, 5B, 5C and 5D were analyzed to confirm that the right PCs were selected for the calibration model. The PRESS represents the sum of the predicted residual error square for the validation and calibration set. With 11 PCs, the validation set had a V-set PRESS value of 10,676.08, and C-set PRESS is 21,252.14. Similarly, with 11 PCs, the values were 11.14 for V set SEE (SEP) and 11.08 for C set SEE (SEC), which were similar and minimal, and it showed the right number of PCs were selected for the model.

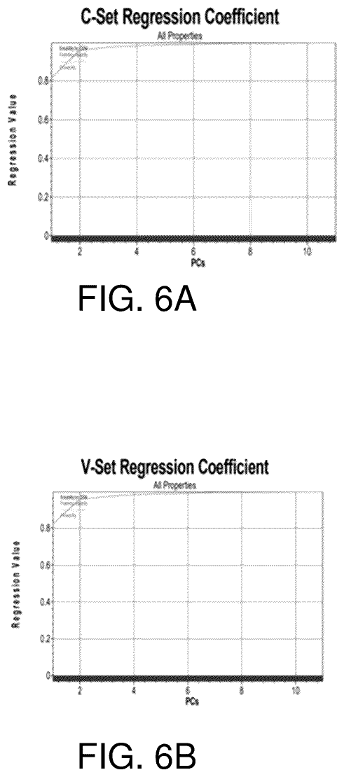

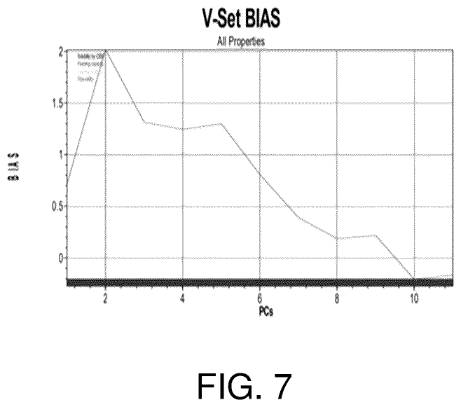

The regression coefficient graphs (FIGS. 6A and 6B) showed how accurately the predicted values match the reference data for the foaming capacity of dairy powders. The V-set regression coefficient was 0.9958, and C-set regression coefficient was 0.9958, which is close to 1, confirming that the accuracy of prediction for foaming capacity was higher with 11 PCs. To understand the precision of the calibration model, the V-set BIAS graph (FIG. 7) was analyzed. With 11 PCs, the V-set BIAS is −0.1648, which was close to zero, demonstrating that the precision of the model was good. Analyzing these graphs provides a summary of the primary PC's selection criteria and also assists in creating a robust model for foaming capacity.

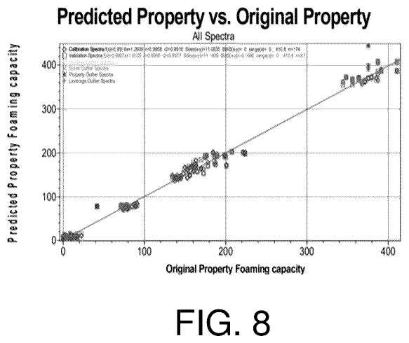

During the development of the prediction model, the predicted vs. original property graphs are analyzed. From the graph shown in FIG. 8, the regression coefficient R² improved after pretreatment with SNV. For foaming capacity, the prediction model developed had an R² value and standard deviation (SD) of 0.9916 and 11.08 for calibration, and the validation sets had a value of 0.9917 and 11.14. This model was selected as an optimal prediction for the foaming capacity of dairy powders in this study.

### NIR Calibration for Foaming Stability

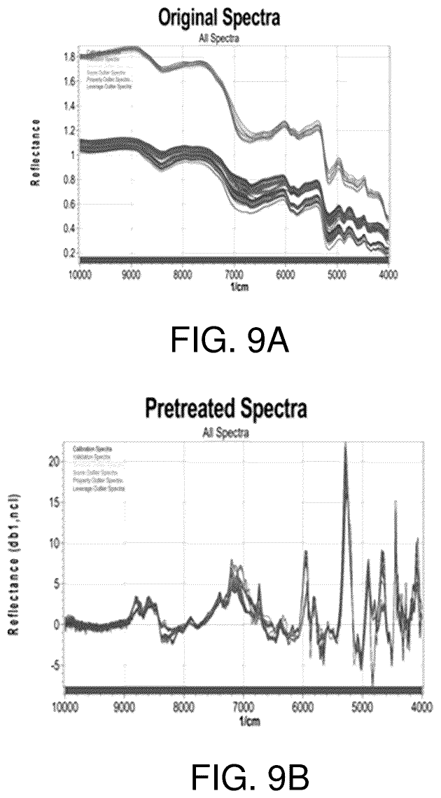

Similar to foaming capacity, several pretreatments reduce inconsistencies and improve the accuracy of prediction with foaming stability. Among the pretreatments for foaming stability, normalization and derivatives work the best in combination for better model prediction of foaming stability. The transformation is performed by 1^{st} derivative BCAP (db1) with linear coefficients 1, 1, 0,−1,−1, 4, and the data is normalized by Closure (ncl). Similar to SNV, normalization by Closure is wavelength-dependent pretreatment. Normalization is used as a pretreatment to reduce baseline variations in the spectra data collected for foaming stability. On the other hand, a derivative is used to reduce the baseline and increase smaller absorption peaks. FIG. 9A shows the spectra without pretreatment and FIG. 9B shows the spectra after 1^{st} derivative BCAP and normalization by Closure is applied as pretreatment for foaming stability.

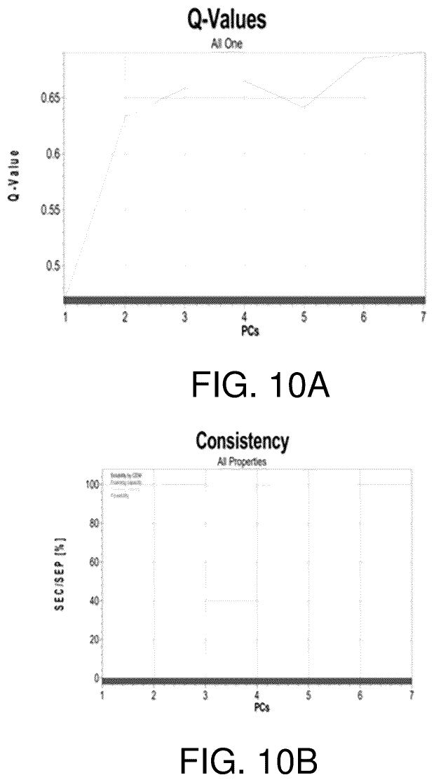

The number of principal components selected for calibration directly influence the model. For one embodiment, 7 principal components are selected as optimal for calibration of the foaming stability of dairy powders. Furthermore, the Q value, which is a Proprietary quality factor from Büchi, provided a value of 0.69 for 7 PCs with pretreatment (FIG. 10A), which served as a quality marker to determine the optimal number of PCs for the dataset. Also, consistency graphs supported an understanding of the relationship between SEC and SEP, and with 7 PCs, the consistency was 99%, as shown in FIG. 10B.

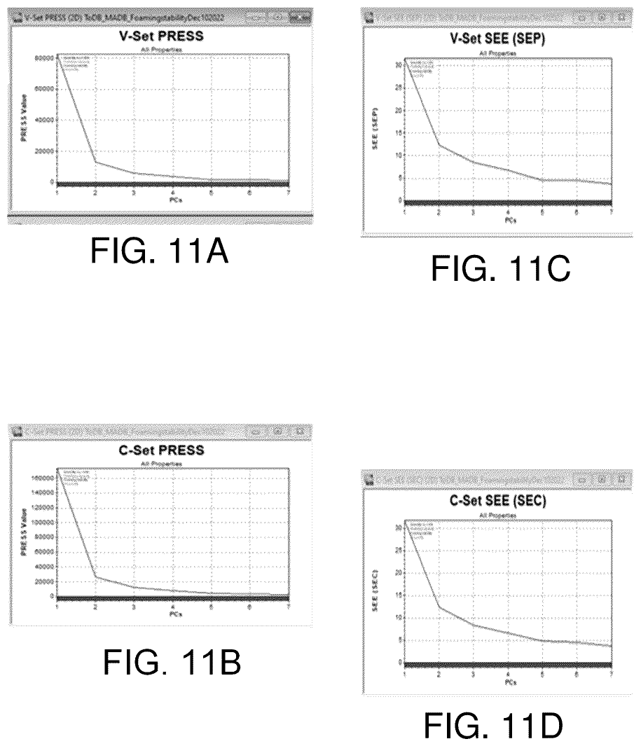

The graphs from FIGS. 11A, 11B, 11C and 11D confirm that the number of PCs was optimum for the model. From the graphs, after pretreatments, 7 PCs were selected. With 7 PCs, the validation set had a V-set PRESS value of 1119.0627, and a C-set PRESS value of 2383.7122 and the values for V-set SEE (SEP) were 3.6704 and for C-set SEE (SEC) were 3.7227, which were similar and minimal, and it shows the right number of PCs were selected for the model.

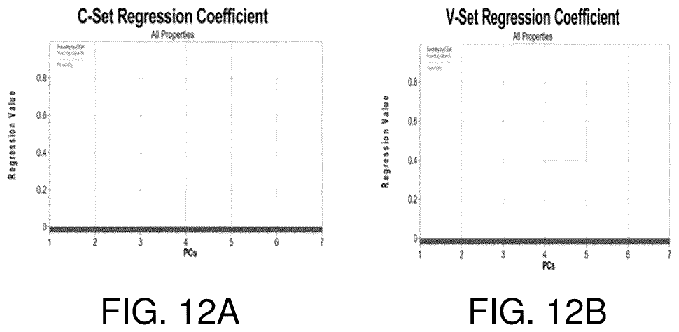

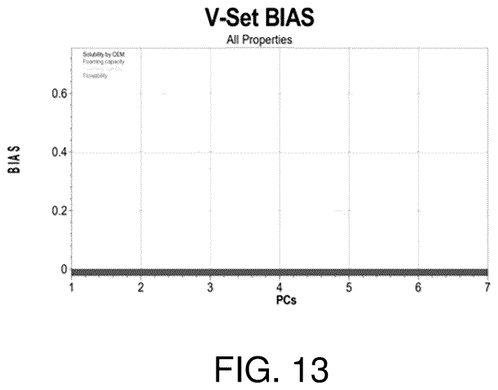

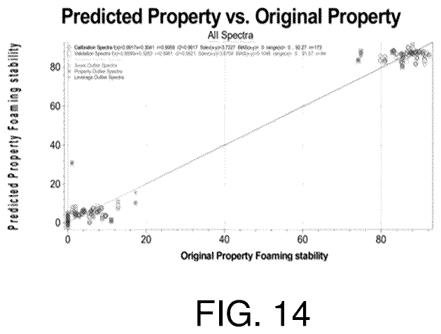

With 7 PCs, the V-set regression coefficient was 0.9961, and C-set regression coefficient was 0.9959 (FIGS. 12A and 12B). The V-set BIAS graph (FIG. 13) indicates the model's precision. With 7 PC's, the V-set BIAS was 0.1048, which was close to zero, demonstrating that the precision of the model is good. From the graphs shown in FIG. 14, the regression coefficient R² improved after pretreatment to 0.9917 with SD 3.72 for calibration and 0.9921 with SD 3.67 for the validation set for foaming stability. This was selected as an optimal prediction for the foaming stability of dairy powders.

### NIR Calibration for Solubility

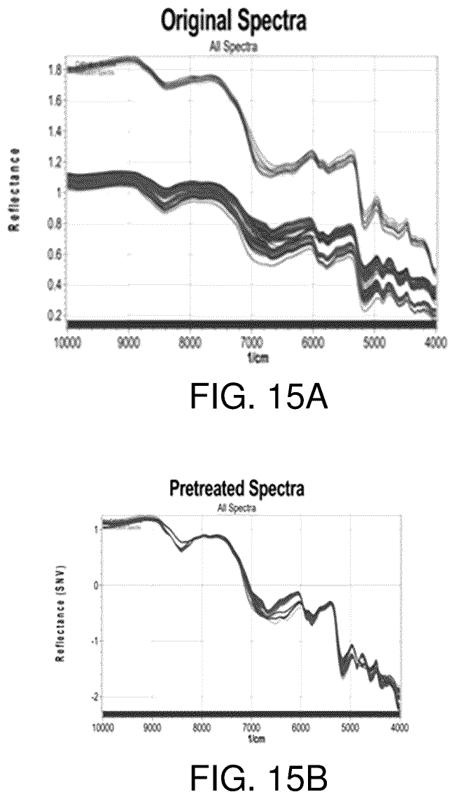

To reduce the inconsistencies and improve prediction accuracy, several pretreatments were used for the solubility model. Among the pretreatments, normalization with standard normal variate works the best in combination for better prediction for solubility. Similar to the model for foaming capacity, normalization by SNV is used to reduce the scattering effects thereby minimizing the noise. FIG. 15A and 15B show the spectra with and without pretreatment.

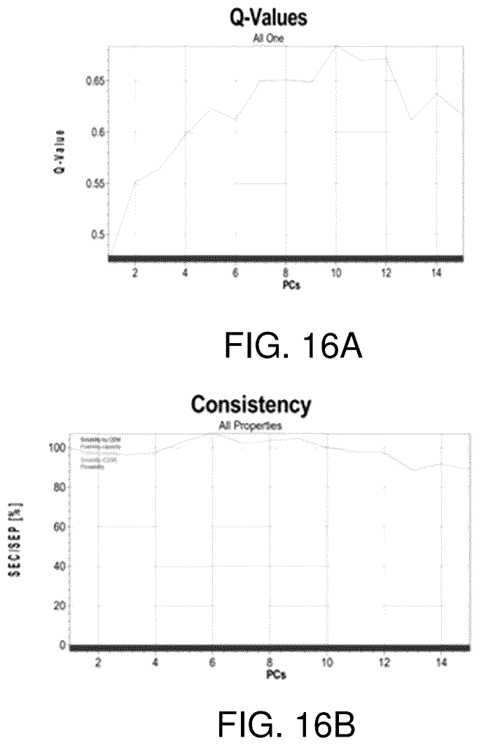

Since PCs directly influence the calibration model, the optimum number of 15 PC's was selected. From FIG. 16A, the Q value for the model was 0.6175, and consistency is 99%, as shown in FIG. 16B, proving the robustness of the model and selection for the optimum number of PCs.

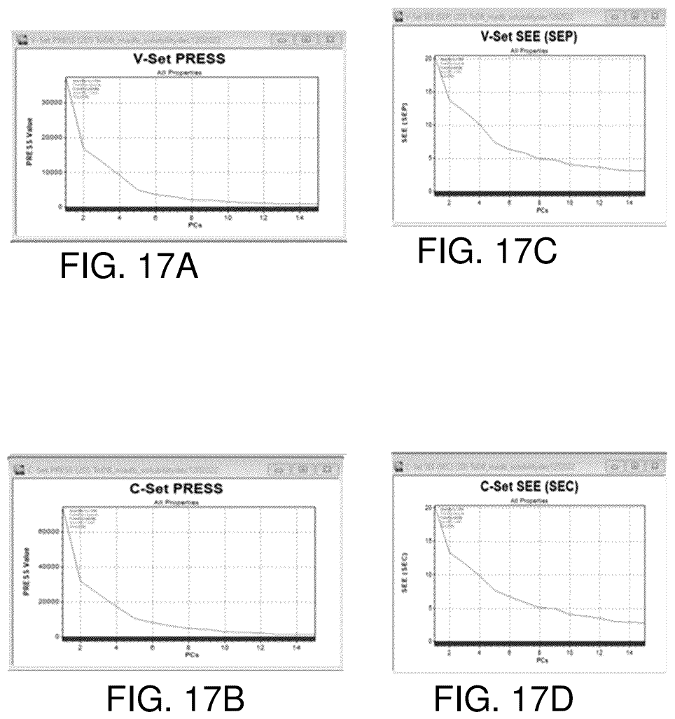

The graphs from FIGS. 17A, 17B, 17C and 17D the PCs for the model, and 15 PCs were selected. With 15 PCs, the validation set has a V-set PRESS 873.8284 value, and the calibration set has a C-set PRESS of 1392.3349, and the values for V-set SEE (SEP) is 3.1326 and for C-set SEE (SEC) is 2.789 which were similar and minimal, and it shows the right number of PCs were selected for the model.

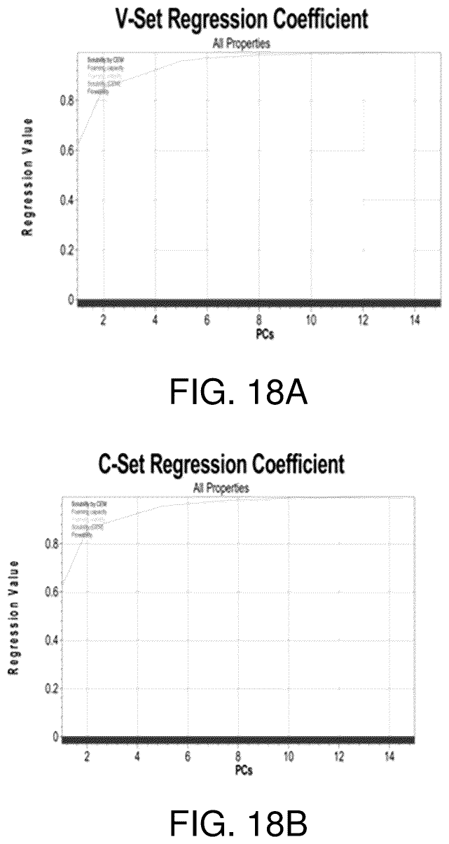

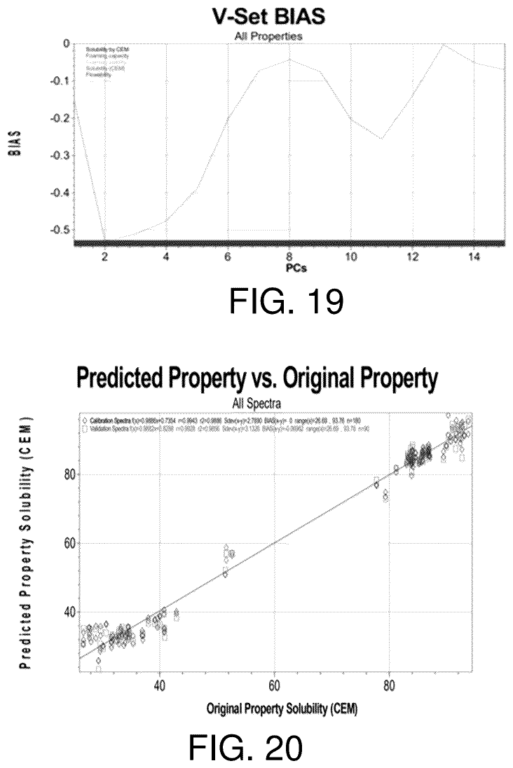

The V-set BIAS graph (FIG. 19) shows the model's precision. With 15 PCs, the V-set regression coefficient is 0.9928, and C-set regression coefficient is 0.9943 (FIGS. 18A and 18B). V-set BIAS is −0.0696, which was close to zero, demonstrating that the precision of the model was good. From the graph shown in FIG. 20 between the original vs. predicted property, the regression coefficient R² improved after pretreatment, 0.9886 with an SD of 2.789 for calibration and 0.9856 with an SD of 3.1326 for the validation set.

In addition to the pretreatments, another critical step in spectral data analysis is removing any uninformative wavelengths or variables creating excessive noise using principal component analysis.

FIG. 21 provides a block diagram of a system containing a spectrophotometer 2100 consisting of spectral analyzer 2101, a microprocessor 2102, a memory 2104, a light detector 2106, light emitter 2108, interferometer 2110, a sample cup 2114, a turntable 2116, a motor 2118, and a network interface 2120.

Microprocessor 2102 is connected to network interface 2120 and communicates with other computing devices on a network 2140 through network interface 2120. Microprocessor 2102 is also connected to memory 2104 and is able to store and retrieve data, configuration settings and instructions from memory 2104.

Spectral analyzer 2101 is connected to microprocessor 2102, light detector 2106 and light emitter 2108. Spectral analyzer 2100 receives configuration settings and instructions from microprocessor 2102 and returns the results of spectral analyses to microprocessor 2102. Spectral analyzer 2101 also provides control signals to light emitter 2108 to cause them to emit light during spectral analysis and receives electrical signals from light detector 2106 representing the light reflected from the sample.

Microprocessor 2102 is also connected to motor 2118 and is able to control the rotation of motor 2118. During spectral analysis, microprocessor 2102 activates motor 2118 thereby causing motor 2118 to rotate turntable 2116 and sample cup 2114.

Computer 2130 includes a microprocessor 2132, a memory 2134, a network interface 2136, a display 2138, a display interface 2142, an input device 2144 and an input device interface 2146. Microprocessor 2132 is connected to memory 2134 and is able to read and write data and instructions to and from memory 2134. Microprocessor 2132 is also connected to network interface 2136 and is able to communicate with other devices through network interface 2136, including spectrophotometer 2100 and a server 2160. Microprocessor 2132 displays user interfaces on display 2138 by sending signals through display interface 2142 and is able to receive input signals from input device 2144 through input device interface 2146. In accordance with one embodiment, input device 2144 is a keyboard. In other embodiments, display 2138 is a touch screen and microprocessor 2132 is able to receive input signals from display 2138 through display interface 2142.

After placing the sample 2122 in spectrophotometer 2100, a user of computer 2130 selects a control displayed in a user interface provided by microprocessor 2132 on display 2138. In accordance with one embodiment, the control allows the user to initiate a spectral analysis of the sample in spectrophotometer 2100. Based on the user's input, microprocessor 2132 sends an instruction to microprocessor 2102 of spectrophotometer 2100 to perform a spectral analysis. In response, microprocessor 2102 sends control signals to motor 2118 to cause it to start rotating turntable 2116 and sample cup 2114. Microprocessor 2102 then instructs spectral analyzer 2101 to perform a spectral analysis of sample 2122. During the spectral analysis, spectral analyzer 2101 causes light emitter 2108 to emit light across a spectrum. In one embodiment, the spectrum includes near infrared light. The light from light emitter 2108 passes through interferometer 2110 which creates a delayed version of the light from light emitter 2108 and causes the delayed version to interfere with the original light from light emitter 2108. The amount of delay is changed over time thereby creating a time-varying light signal at the output of interferometer 2110. The time-varying light impinges sample 2122 and some wavelengths of the light are reflected by sample 2122 while other wavelengths of the light are absorbed by sample 2122. The light reflected by sample 2122 passes to light detector 2106 where the light is converted into an electrical signal that indicates the intensity of the light received at detector 2106. Because the light from light emitter 2108 is a time-varying light signal, the light received by detector 2106 is also a time-varying signal resulting in a time-varying electrical signal at the output of detector 2106.

The time-varying electrical signal is provided to spectral analyzer 2101, which performs a Fourier Transform of the time-varying electrical signal to generate intensity values for various spectral bands of light in the time-varying light signal received by detector 2106. Together, light detector 2106 and spectral analyzer 2101 form a light sensing assembly. Spectral analyzer 2101 then either sends the amount of reflected light at each spectral band or the amount of light absorbed by the sample at each spectral band to microprocessor 2102. The amount of light absorbed or the amount of light reflected for a particular spectral band is referred to herein as a spectral value. In accordance with one embodiment, the amount of light absorbed at each spectral band is determined by subtracting the amount of reflected light at the spectral band from a maximum amount of light reflected for any spectral band.

Each spectral band contains a continuous segment of different wavelengths of light. The number of spectral bands, the width of each band and any separation between the bands is typically fixed in the spectrophotometer or can be adjusted through configuration settings.

Microprocessor 2102 returns the spectral values it receives from spectral analyzer 2100 to microprocessor 2132. During construction of a model, microprocessor 2132 uses the spectral values and a value for a functional property of the sample to identify which spectral bands are principal components for describing a relationship between a near infrared reflectance spectrum and a functional property of dairy powders. Specifically, measured values for the functional property of dairy powder samples are used with the spectral values to identify which spectral bands are correlated to the functional property and the relationship between the intensity of the spectral band and the value of the functional property to provide a model that can predict a value for the functional property given the spectral values for a sample.

During use of the model, microprocessor 2132 applies the spectral values to the model to generate a predicted value for the functional property of the dairy powder.

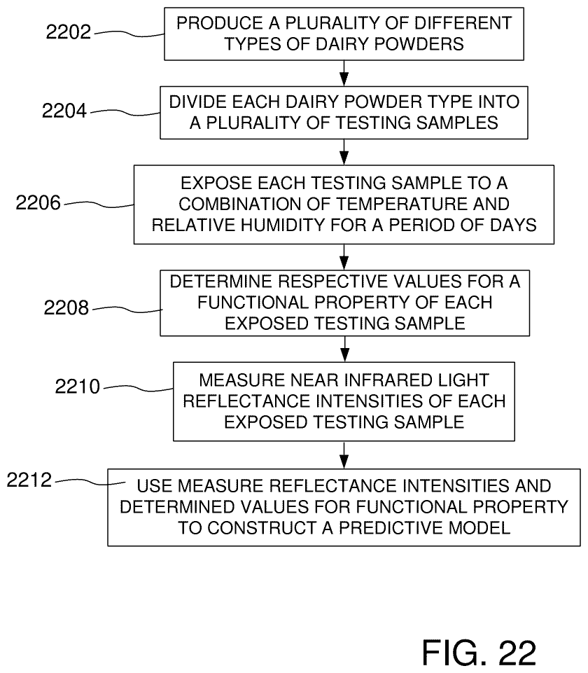

FIG. 22 provides a flow diagram of a method of forming a predictive model in accordance with one embodiment. In step 2202, a plurality of different types of dairy powder are produced including at least two of: whey protein isolate, whey protein concentrate, milk protein isolate, milk protein concentrate, and nonfat dry milk. In step 2204, each dairy powder type is divided into a plurality of testing samples and at step 2206, each testing sample is exposed to a combination of temperature and relative humidity for a period of days. In one embodiment, eighteen testing samples are formed for each dairy powder type and eighteen combinations of temperature, relative humidity and exposure time are used in step 2206 with one testing sample exposed to each combination of temperature, relative humidity and exposure time as shown in Table 1 above.

At step 2208, a value is determined for a functional property of each of the exposed testing samples such as a value for solubility, foaming capacity or foaming stability. At step 2210, spectrophotometer is used to determine near infrared light reflectance intensities for each exposed testing sample. At step 2112, the determined values for the functional property and the reflectance intensities are used to construct a predictive model by identifying which spectral bands are principal components in predicting the values for the functional properties and the relationship between those intensities and the values of the functional property.

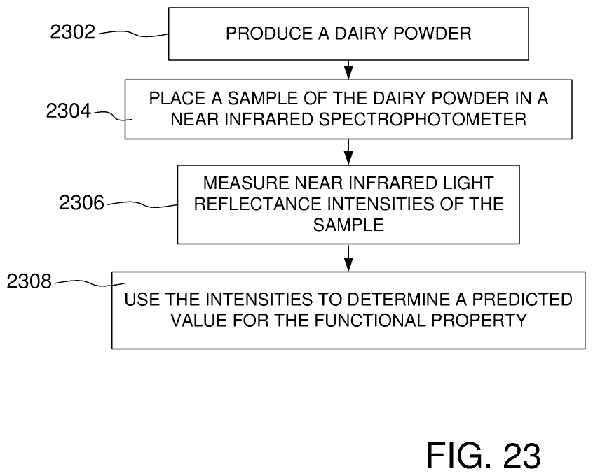

FIG. 23 provides a flow diagram for predicting a value for a functional property of a dairy powder. In step 2302, the dairy powder is formed. In step 2304, a sample of the dairy powder is placed in a near infrared spectrophotometer and at step 2306 intensities of different wavelengths of light reflected from the sample are determined. In accordance with one embodiment, the wavelengths of light are within the near infrared band of light. In step 2308, the determined intensities are used to determine a predicted value for the functional property. In accordance with one embodiment, the predicted value is determined by applying the intensities to a model that has been trained on data from a plurality of different dairy powder types that have been exposed to a plurality of combinations of temperature and relative humidity for a plurality of different durations.

Although elements have been shown or described as separate embodiments above, portions of each embodiment may be combined with all or part of other embodiments described above.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms for implementing the claims.

What is claimed is:

1. A method of predicting a value for a functional property of a dairy powder, the method comprising:
placing a sample of the dairy powder in a near-infrared spectrophotometer;
using the near-infrared spectrophotometer to determine intensities of different wavelengths of light reflected from the sample within a near-infrared band; and
using the intensities to determine a predicted value for the functional property.

2. The method of claim 1 wherein the functional property comprises a solubility of the dairy powder.

3. The method of claim 2 wherein the functional property comprises a foaming capacity of the dairy powder.

4. The method of claim 3 wherein the functional property comprises a foaming stability of the dairy powder.

5. The method of claim 4 wherein using the intensities to determine a predicted value for the functional property comprises using a model trained on a plurality of dairy powder types.

6. The method of claim 5 wherein the plurality of dairy powder types comprises at least two of: whey protein isolate, whey protein concentrate, milk protein isolate, milk protein concentrate, and nonfat dry milk.

7. The method of claim 6 wherein using a model trained on a plurality of dairy powder types further comprises using a model trained on a plurality of dairy powder types exposed to a plurality of different humidity and temperature combinations.

8. A dairy powder functional prediction system comprising:
a near-infrared light source providing wavelengths of light across a near-infrared spectrum;
a sample holder for holding a sample of a dairy powder;
a light sensing assembly positioned to measure intensities of different wavelengths of light reflected by the dairy powder in the sample holder; and
a processor, receiving the measured intensities and using the measured intensities to predict a value for a functional property of the dairy powder.

9. The dairy powder functional prediction system of claim 8 wherein the functional property comprises a solubility of the dairy powder.

10. The dairy powder functional prediction system of claim 8 wherein the functional property comprises a foaming capacity of the dairy powder.

11. The dairy powder functional prediction system of claim 8 wherein the functional property comprises a foaming stability of the dairy powder.

12. The dairy powder functional prediction system of claim 8 wherein using the measured intensities to predict a value for the functional property comprises using a model trained on a plurality of dairy powder types.

13. The dairy powder functional prediction system of claim 8 wherein the plurality of dairy powder types comprises at least two of: whey protein isolate, whey protein concentrate, milk protein isolate, milk protein concentrate, and nonfat dry milk.

14. The dairy powder functional prediction system of claim 8 wherein using a model trained on a plurality of dairy powder types further comprises using a model trained on a plurality of dairy powder types exposed to a plurality of respective different humidity and temperature combinations.

15. A method comprising:
producing a plurality of different types of dairy powders;
dividing each dairy powder type into a plurality of testing samples;
exposing each testing sample to a respective temperature for a period of days;
measuring near-infrared light reflectance intensities of the exposed testing samples;
determining respective values for a functional property of each exposed testing sample; and
using the measured reflectance intensities and the determined values for the functional properties to construct a model that predicts values for the functional property from measured near-infrared light reflectance intensities.

16. The method of claim 15 wherein at least two of the testing samples are exposed to different respective temperatures.

17. The method of claim 16 further comprising exposing each testing sample to a relative humidity, wherein at least two of the testing samples are exposed to different respective relative humidities.

18. The method of claim 15 wherein the functional property comprises a solubility of the dairy powder.

19. The method of claim 15 wherein the functional property comprises a foaming capacity of the dairy powder.

20. The method of claim 15 wherein the functional property comprises a foaming stability of the dairy powder.
